(19) United States
(12) Patent Application Publication
(10) Pub. No.: US 2021/0270685 A1
(43) Pub. Date: Sep. 2, 2021

(54) OPTICAL FIBER SENSING MONITORING DEVICE FOR SOIL SETTLEMENT AND SETTLEMENT AMOUNT MEASUREMENT METHOD

(71) Applicant: Shenzhen University, Shenzhen (CN)

(72) Inventors: Chengyu Hong, Shenzhen (CN); Xiangsheng Chen, Shenzhen (CN); Dong Su, Shenzhen (CN); Junkun Tan, Shenzhen (CN); Wenli Liu, Shenzhen (CN)

(73) Assignee: Shenzhen University

(21) Appl. No.: 17/325,191

(22) Filed: May 19, 2021

(30) Foreign Application Priority Data
Apr. 28, 2021 (CN) 202110466421.3

(51) Int. Cl. G01L 1/24; G01N 33/24; E02D 1/02

(52) U.S. Cl. CPC G01L 1/242; E02D 1/022; G01N 33/24

(57) ABSTRACT

The present disclosure discloses an optical fiber sensing monitoring device for soil settlement and a settlement amount measurement method. The optical fiber sensing monitoring device includes a baseline rod that is internally hollowed. A plurality of settlement monitoring circular rings are sleeved outside the baseline rod, and are coaxial with the baseline rod; each settlement monitoring circular ring is provided with one trigger device that can spread an anchor by means of touching; the baseline rod is provided with through holes corresponding to the settlement monitoring circular rings; the trigger devices are provided with trigger sticks on the inner sides; and the trigger sticks extend into the baseline rod via the through holes.

### TECHNICAL FIELD

The present disclosure relates to the technical field of civil engineering monitoring equipment, and in particular, an optical fiber sensing monitoring device for soil settlement and a settlement amount measurement method.

### BACKGROUND

Usually, a flexible tape settlement meter is used to monitor a layered settlement amount of a soil foundation. A working method of the flexible tape settlement meter is as follows: a PVC center-through settling tube is pre-buried in a soil foundation to be tested, and settling magnetic rings are sectionally sleeved outside the center-through settling tube; a bottom port of the bottommost end of the settling tube is provided with a conical guide pipe sealing head; there are a plurality of settlement monitoring circular rings distributed on the outer surface of the settling tube; a magnetic ring is arranged in the settlement monitoring circular ring; a radially outward anchor iron sheet that can be retracted is connected outside the settlement monitoring circular ring; and the anchor iron sheet is embedded in the tested soil layer such that the settling magnetic rings settle with the tested soil layer.

Before being buried, the anchor iron sheet of the traditional settlement monitoring circular ring is strongly folded and tied to the side wall of the center-through settling tube with a water-soluble paper tape. In the process of mounting the center-through settling tube into a monitoring hole, the settlement monitoring circular ring easily moves and cannot be applied to soil layers with insufficient moisture.

### SUMMARY

The purpose of the present disclosure is to solve the defects in the existing technology to provide an optical fiber sensing monitoring device for soil settlement and a settlement amount measurement method, thereby solving the problems in the existing technology.

In order to achieve the foregoing purpose, the present disclosure adopts the following technical solution:

an optical fiber sensing monitoring device for soil settlement, including a baseline rod that is internally hollowed. An outer ring of the baseline rod is fixedly connected with a plurality of optical fiber sensors in a ring array manner by taking the baseline rod as a baseline; a plurality of settlement monitoring circular rings are sleeved outside the baseline rod, and are coaxial with the baseline rod; each settlement monitoring circular ring is provided with one trigger device that can spread an anchor by means of touching; the baseline rod is provided with through holes corresponding to the settlement monitoring circular rings; the trigger devices are provided with trigger sticks on the inner sides; and the trigger sticks extend into the baseline rod via the through holes.

The optical fiber sensing monitoring device further includes a stop lever that can extend into an inner tube of the baseline rod and stop the settlement monitoring circular rings.

Preferably, the baseline rod is one of a PVC tube or an AGR tube.

Preferably, the baseline rod is provided with mark lines that correspondingly mark the positions of the trigger sticks at the upper end.

Preferably, the upper end of the stop lever is fixedly connected with a circular truncated cone, and the circular truncated cone is provided with an alignment line on the upper side.

Preferably, the outer sides of the optical fiber sensors are fixedly connected with a rubber sleeve.

Preferably, a spiral groove is formed in the outer side of the rubber sleeve.

A soil settlement amount monitoring method that uses the aforementioned monitoring device includes the following operation steps:

at a first step: smearing an orifice of the baseline rod with lubricating grease, symmetrically placing two cushion blocks with a height of 50-100 mm at the upper end of the baseline rod, constantly lowering the stop lever in a manner of avoiding the trigger sticks by referring to the mark lines, and after the circular truncated cone of the stop lever is in contact with the upper end of the baseline rod, rotating the stop lever such that the alignment line arranged on the upper side of the circular truncated cone is aligned with the mark line;

at a second step: drilling a monitoring hole in a position to be measured in a manner that the depth of the monitoring hole reaches the position of a rigid rock stratum to form an insertion hole in the rigid rock stratum, digging a plurality of radial trenches in the stratum surface by taking the monitoring hole as a baseline, putting the baseline rod into the monitoring hole such that the optical fiber sensors correspondingly enter the trenches, and embedding the lower end of the baseline rod into the insertion hole of the rigid rock stratum;

at a third step: using the stop lever to touch and spread an anchor after the cushion blocks are removed, embedding the anchor into the soil layer, and taking out the stop lever; and

at a fourth step: an operator putting the magnetic induction probe end of a professional measurement flexible tape into the baseline rod and holding the flexible tape with a hand to let the magnetic induction probe to slowly move downwards; a receiver making a prompt tone from the inside when the magnetic induction probe reaches a position for burying the settlement monitoring circular rings in the soil layer; at this time, artificially reading a depth size of the measurement flexible tape in the baseline rod; and comparing this measured value with a measured value of the previous time point to obtain a settlement value of the soil layer with the settlement monitoring circular rings within this period of time.

Preferably, the trenches are filled with a backfill.

The present disclosure has the advantages that in the optical fiber sensing monitoring device for soil settlement and the settlement amount measurement method, the long axis of an elliptical turntable will push sliders to the outside by rotating the elliptical turntable, and the sliders extend into insertion holes for limitation, so that an annular sleeve will not move as the baseline rod moves downwards and is applicable to a soil layer with insufficient moisture; and meanwhile, deformation caused by spreading of arc-shaped plates due to the displacement of the settlement monitoring circular rings can be reduced, so that a measured settlement amount is closer to a theoretical value.

### BRIEF DESCRIPTION OF THE DRAWINGS

FIG. 1 is a schematic diagram of a basic structure of the present disclosure;

FIG. 2 is a partially enlarged diagram of a portion E in FIG. 1;

FIG. 3 is a schematic structural diagram of a stop lever in the present disclosure;

FIG. 4 is a partially enlarged diagram of a portion F in FIG. 3;

FIG. 5 is a cutaway view along A-A in FIG. 4;

FIG. 6 is a schematic diagram of a connection structure of a hollow settling tube and a stop lever in the present disclosure;

FIG. 7 is a schematic structural diagram of a settlement monitoring circular ring in the present disclosure;

FIG. 8 is a schematic structural diagram after an arc-shaped plate of a settlement monitoring circular ring in the present disclosure is spread;

FIG. 9 is a partially enlarged diagram of a portion G in FIG. 8;

FIG. 10 is a partially enlarged diagram of a portion H in FIG. 9;

FIG. 11 is a schematic diagram of a connection structure of an arc-shaped plate in the present disclosure;

FIG. 12 is a schematic diagram of a rotation principle of an arc-shaped plate in the present disclosure; and

FIG. 13 is a layout chart of trenches in the present disclosure.

### DESCRIPTION OF THE EMBODIMENTS

In order to make the objectives, technical solutions and advantages of the present invention clearer, the present invention is further described below in detail with reference to accompanying drawings and embodiments. It should be understood that the specific embodiments described here are merely to explain the present disclosure, and not intended to limit the present disclosure.

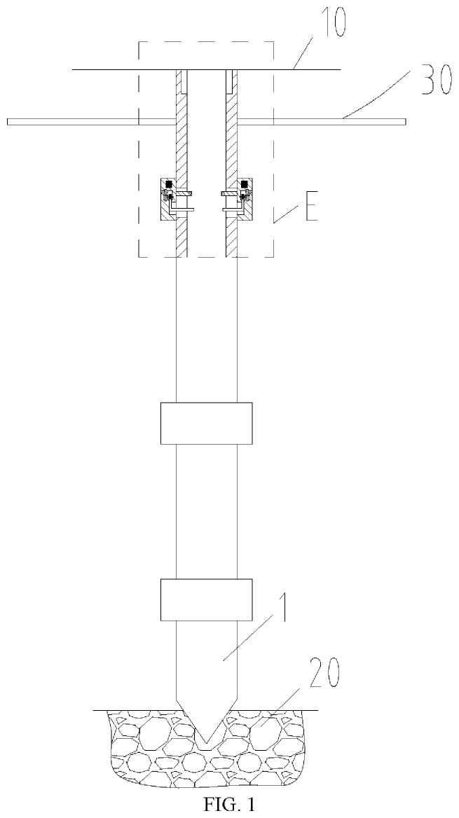

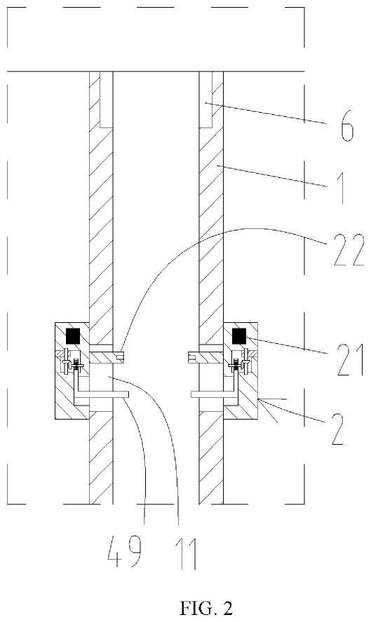

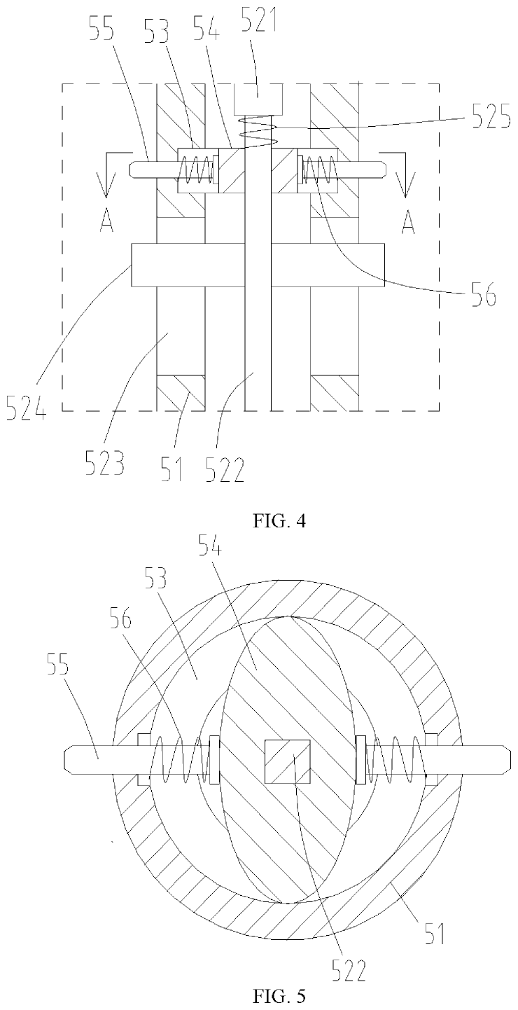

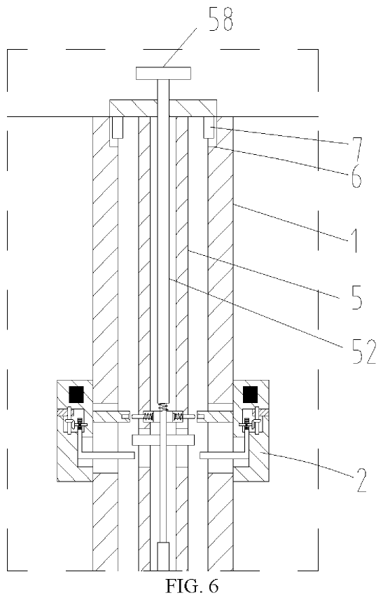

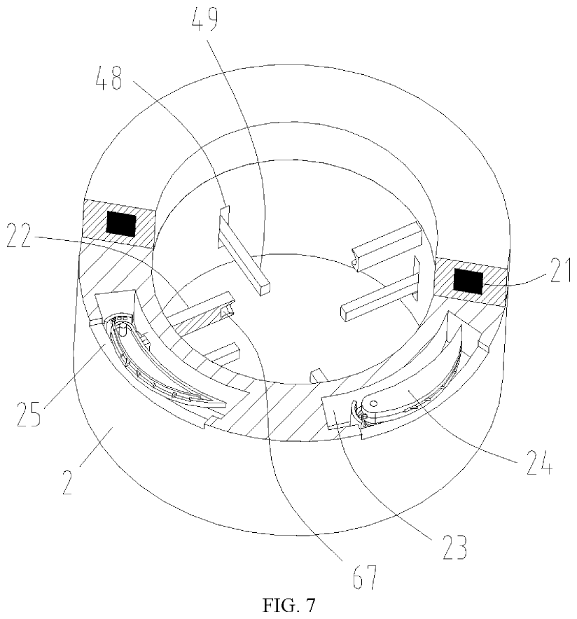

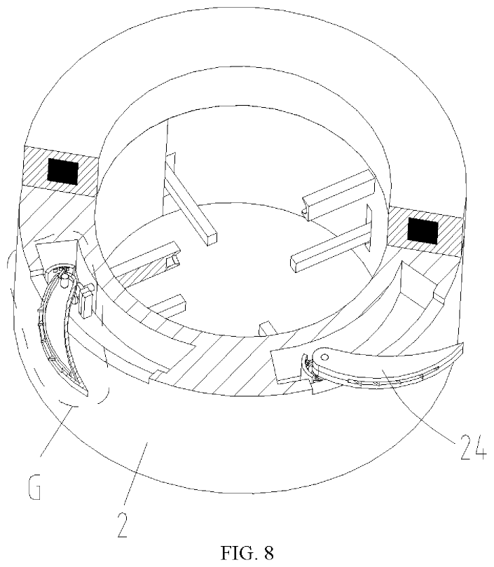

As shown in FIG. 1 to FIG. 13, an optical fiber sensing monitoring device for soil settlement provided by the present disclosure includes an internally hollowed baseline rod 1 that is one of a PVC tube or an AGR tube; and the outer ring of the baseline rod 1 is fixedly connected with a plurality of optical fiber sensors 30 in a ring array manner by taking the baseline rod 1 as a baseline. The optical fiber sensors 30 are the existing technology, the basic working principle of which is to feed light from a light source to a modulator such that parameters to be measured and the light that enters a modulation region act with each other to cause the optical properties (such as the intensity, the wavelength, the frequency, the phase and the polarization state) of the light to change, and the light is referred to as modulated signal light; and measurement is completed by using the influence of the measured soil on the transmission characteristics of the light. The outer sides of the optical fiber sensors 30 are fixedly connected with a rubber sleeve that can deform together with the optical fiber sensors 30 inside it. The outer side of the rubber sleeve is provided with a spiral groove. The spiral groove of the rubber sleeve is conductive to tight embedding to the soil. A plurality of settlement monitoring circular rings 2 are sleeved outside the baseline rod 1; the inner tube walls of the settlement monitoring circular rings 2 are provided, in an embedded manner, with magnet rings 21 used to trigger a magnetic induction element of a probe of a settlement meter; the settlement monitoring circular rings 2 are coaxial with the baseline rod 1. Each settlement monitoring circular ring 2 is provided with one trigger device that can touch and spread an anchor. The anchor is a rotatable arc-shaped plate 24. A mounting cavity 23 is formed inside each settlement monitoring circular ring 2, and the arc-shaped plate 24 is rotatably connected into the mounting cavity 23. The outer sides of the settlement monitoring circular rings 2 are provided with notches 25 communicating with the mounting cavities 23. The arc-shaped plate 24 can rotatably extend out from the notches 25. The arc-shaped plate 24 is rotatably embedded into the soil layer to measure layered settlement of the soil.

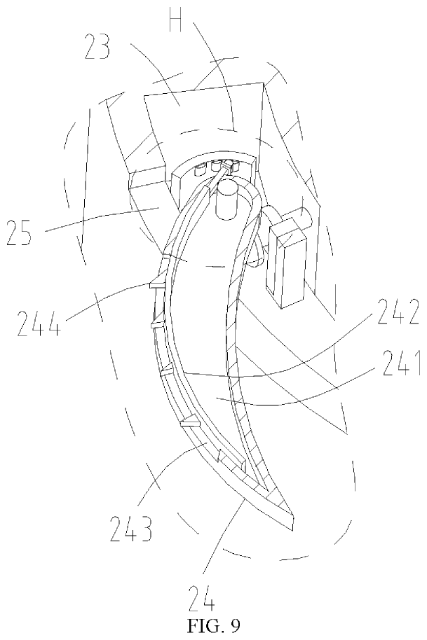

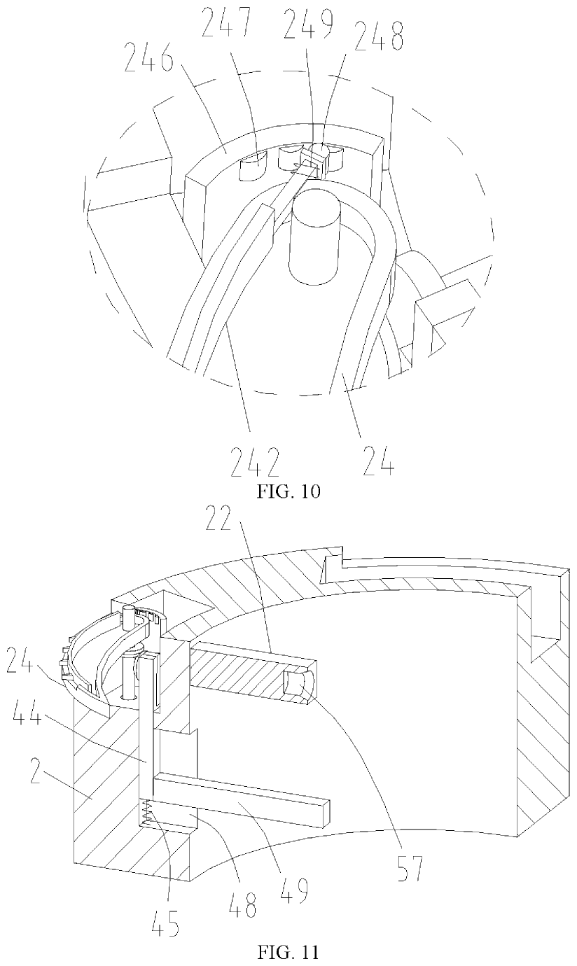

As shown in FIG. 9 to FIG. 10, the arc-shaped plate 24 is provided with a collection cavity 241; the inner wall of a side of the collection cavity 241 close to an outer arc of the arc-shaped plate 24 is slidably connected with an arc-shaped strip 242; one side of the outer arc of the arc-shaped plate 24 is provided with a yield slot 243; the arc-shaped strip 242 is provided with sawteeth 244 on the outer arc surface; the sawteeth 244 extend out from the yield slot 243; the arc-shaped plate 24 is provided with a push rod 245 on one side; the push rod 245 drives the arc-shaped strip 242 to slide on the inner wall of the collection cavity 241 in a reciprocating manner; an arc slab 246 is fixedly connected to the inside of the mounting cavity 23; a plurality of lugs 247 are arranged on the inner arc surface of the arc slab 246 at intervals; the end socket of the push rod 245 is rotatably connected with a pin roller 248; a first spring 249 is arranged between a mounting plate of the pin roller 248 and the arc-shaped plate 24; in the process that the arc-shaped plate 24 rotates, the pin roller 248 rolls on the arc slab 246; the arc-shaped strip 242 moves in a reciprocating manner through cooperation between the lugs 247 and the first spring 249 to saw the soil layer and reduce the resistance to embedding the arc-shaped plate 24 into the soil layer;

The baseline rod 1 is provided with through holes 11 corresponding to the settlement monitoring circular rings 2; each through hole 11 communicates with an inner cavity of the baseline rod 1; inner rings of the settlement monitoring circular rings 2 are provided with a plurality of supporting rods 22 at intervals; each supporting rod 22 extends into the inner cavity of the baseline rod 1 through the corresponding through hole 11; and the trigger devices are provided with trigger sticks 49 on the inner sides; and the trigger sticks 49 extend into the baseline rod 1 through the through holes 11.

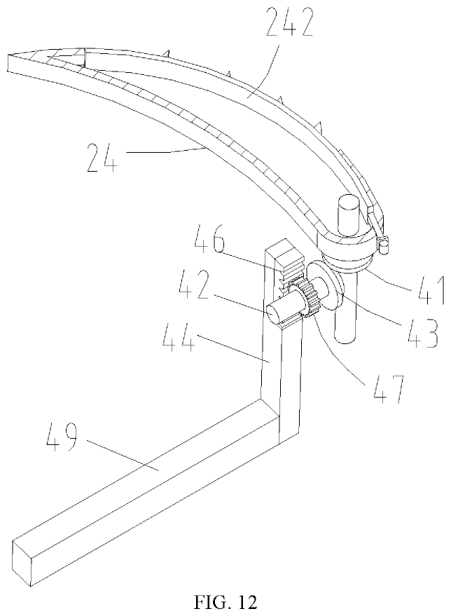

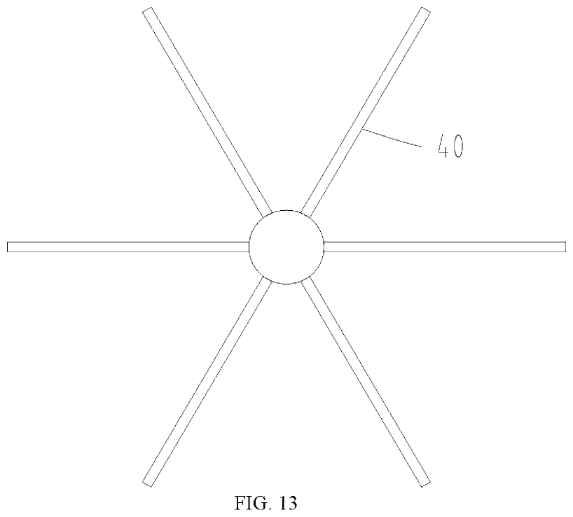

As shown in FIG. 11 to FIG. 12, the trigger device includes a first bevel gear 41 that is fixedly connected to a rotating shaft of the arc-shaped plate 24; the side wall of the mounting cavity 23 is rotatably connected with a driving shaft 42; the end socket of the driving shaft 42 is fixedly connected with a second bevel gear 43; the second bevel gear 43 meshes the first bevel gear 41; the mounting cavity 23 is provided with a sliding hole in the bottom; a square rod 44 is slidably connected into the sliding hole; a supporting spring 45 is fixedly connected between the square rod 44 and the bottom of the sliding hole; one side of the upper end of the square rod 44 is fixedly connected with a toothed plate 46; the middle part of the driving shaft 42 is fixedly connected with a driving gear 47; the driving gear 47 meshes the toothed plate 46; the inner wall of the settlement monitoring circular ring 2 is provided with a sliding chute 48 that communicates with the sliding hole; the trigger stick 49 is slidably connected into the sliding chute 48 and is fixedly connected to the square rod 44; and one end of the trigger stick 49 extends into an inner tube of the baseline rod 1.

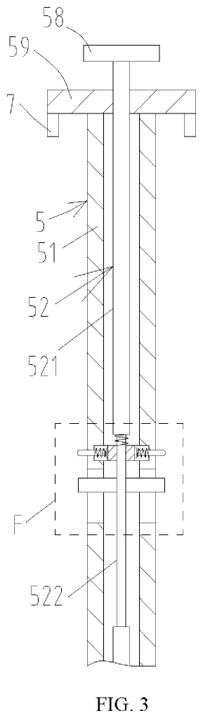

As shown in FIG. 3 to FIG. 6, one stop lever 5 is further included. The stop lever 5 can extend into the inner tube of the baseline rod 1 to stop the settlement monitoring circular rings 2, and is used to trigger the arc-shaped plate 24 to rotate; the trigger stick 49 is pushed to move downwards through the stop lever 5, and then the driving shaft 4 is driven by the toothed plate 46 to rotate; and the arc-shaped plate 24 is spread via transmission of the second bevel gear 43 and the first bevel gear 41. Compared with the existing technology, the present disclosure is driven by a motor to rotate by contacting a touch switch, can achieve the purpose by pushing with a simple mechanism, and is simple in operation and firm;

the stop lever 5 includes a hollow lever body 51; an operating rod 52 is coaxially arranged inside the lever body 51; the lever body 51 is provided with a ring slot 53 on the inner wall; an elliptical turntable 54 is arranged on the operating rod 52, and is rotatably connected into the ring slot 53; the ring slot 53 is provided with one sliding hole in each of two ends; the inside of each sliding hole is slidably connected with one slider 55; each slider 55 is provided with a boss at an end close to the elliptical turntable 54; the boss is in contact with the outer ring surface of the elliptical turntable 54; a second spring 56 is arranged between the boss and the inner wall of the ring slot 53; the supporting rod 22 are provided with insertion holes 57 in the end sockets; and the insertion holes 57 match the sliders 55. In order to prevent displacement of the whole settlement monitoring circular rings 2 in the process of pushing the trigger sticks 49 to move, the elliptical turntable 54 is rotated such that the long axis of the elliptical turntable 54 pushes the sliders 55 to the outside; the sliders 55 extend into the insertion holes 57 for limitation, so that the settlement monitoring circular rings 2 cannot move as the baseline rod 1 moves downwards and are applicable to the soil layer with insufficient moisture; and meanwhile, the settlement monitoring circular rings 2 would not move to reduce the deformation caused by the spreading of the arc-shaped plate 24, so that the measured settlement amount is closer to a theoretical value.

The operating rod 52 includes round rod sections 521 and square rod sections 522 which are disposed alternately; the elliptical turntable 54 is provided with a run-through square hole in the middle; the square rod sections 522 of the operating rod 52 pass through a square hole and are slidably connected; the lever body 51 is provided with a plurality of through slots 523 in an outer circle; the through slots 523 and the through holes 11 are in one-to-one correspondence; the square rod sections 522 of the operating rod 52 are fixedly connected with a plurality of pressing rods 524; each pressing rod 524 correspondingly controls one arc-shaped plate 24; reset springs 525 are fixedly connected between the lower ends of the round rod sections 521 and the upper side of the elliptical turntable 54; and the operating rod 52 is downwards pushed after the sliders 55 extend into the insertion holes 57 for limitation, and the pressing rods 524 on the square rod sections 522 of the operating rod 52 push down the trigger sticks 49.

The upper end of the lever body 51 is fixedly connected with a circular truncated cone 59; the diameter of the circular truncated cone 59 is greater than the diameter of the baseline rod 1; the baseline rod 1 is provided with mark lines that correspondingly mark the positions of the trigger sticks 49 at the upper end; and the circular truncated cone 59 is provided with an alignment line on the upper side. An orifice of the baseline rod 1 is smeared with lubricating grease; two cushion blocks with a height of 50-100 mm are symmetrically placed at the upper end of the baseline rod 1; the lever body 51 is constantly lowered in a manner of avoiding the trigger sticks 49 by referring to the mark lines; after the circular truncated cone 59 at the upper end of the lever body 51 is in contact with the upper end of the baseline rod 1, the lever body 51 is rotated such that the alignment line arranged on the upper side of the circular truncated cone 59 is aligned with the mark line; at this time, the pressing rods 524 are located over the trigger sticks 49; and in-place installation is realized after the cushion blocks are removed.

A clamping slot 6 is formed in the inner wall of the upper end of the baseline rod 1; the circular truncated cone 59 is provided with a clamping plate 7 at the bottom; the clamping plate 7 matches the clamping slot 6; and the circular truncated cone 59, the clamping plate 7 and the clamping slot 6 locate the position of the stop lever 5 in the baseline rod 1, so that the pressing rods 524 can just face the trigger sticks 49 to avoid the influence caused by dislocation on the spreading of the arc-shaped plates 24.

The present disclosure further discloses a soil settlement amount monitoring method that uses the aforementioned monitoring device, including the following operation steps:

at a first step: an orifice of the baseline rod 1 is smeared with lubricating grease; two cushion blocks with a height of 50-100 mm are symmetrically placed at the upper end of the baseline rod 1; the stop lever 5 is constantly lowered in a manner of avoiding the trigger sticks 49 by referring to the mark lines; and after the circular truncated cone 59 of the stop lever 5 is in contact, the stop lever 5 is rotated such that the alignment line arranged on the upper side of the circular truncated cone 59 is aligned with the mark line;

at a second step: a monitoring hole is drilled in a position to be measured in a manner that the depth of the monitoring hole reaches the position of a rigid rock stratum to form an insertion hole in the rigid rock stratum; a plurality of radial trenches are dug in the stratum surface by taking the monitoring hole as a baseline; the baseline rod 1 is put into the monitoring hole such that the optical fiber sensors 30 correspondingly enter the trenches 40 that is filled with a backfill; and the lower end of the baseline rod 1 is embedded into the insertion hole of the rigid rock stratum;

at a third step: the stop lever 5 is used to touch and spread an anchor after the cushion blocks are removed, and the anchor is embedded into the soil layer, i.e., the operating rod 52 is downwards pushed; the pressing rods 524 on the square rod sections 522 of the operating rod 52 push down the trigger sticks 49; the trigger sticks 49 are pushed to move downwards through the stop lever 5, and then the driving shafts 4 are driven by the toothed plates 46 to rotate; the arc-shaped plates 24 are spread via transmission of the second bevel gears 43 and the first bevel gears 41; and the elliptical turntable 54 is rotated, the sliders 55 leave the insertion holes 57, and the stop lever 5 is removed;

at a fourth step: an operator puts the magnetic induction probe end of a professional measurement flexible tape into the baseline rod 1 and holds the flexible tape with a hand to let the magnetic induction probe to slowly move downwards; a receiver makes a prompt tone from the inside when the magnetic induction probe reaches a position for burying the settlement monitoring circular rings 2 in the soil layer; at this time, a depth size of the measurement flexible tape in the baseline rod 1 is artificially read; and this measured value is compared with a measured value of the previous time point to obtain a settlement value of the soil layer with the settlement monitoring circular rings 2 within this period of time.

Although the embodiments of the present disclosure have been shown and described, it will be understood by those of ordinary skill in the art that various changes, modifications, substitutions, and variations can be made to these embodiments without departing from the principle and spirit of the present disclosure. The scope of the present disclosure is defined by the attached claims and their equivalents.

What is claimed is:

1. An optical fiber sensing monitoring device for soil settlement, comprising a baseline rod (1) that is internally hollowed, wherein an outer ring of the baseline rod (1) is fixedly connected with a plurality of optical fiber sensors (30) in a ring array manner by taking the baseline rod (1) as a baseline; a plurality of settlement monitoring circular rings (2) are sleeved outside the baseline rod (1), and are coaxial with the baseline rod (1); each settlement monitoring circular ring (2) is provided with one trigger device that can spread an anchor by means of touching; the baseline rod (1) is provided with through holes (11) corresponding to the settlement monitoring circular rings (2); the inner sides of the trigger devices are provided with trigger sticks (49); the trigger sticks (49) extend into the baseline rod (1) via the through holes (11);
the optical fiber sensing monitoring device further comprises a stop lever (5) that can extend into an inner tube of the baseline rod (1) and stop the settlement monitoring circular rings (2).

2. The optical fiber sensing monitoring device for soil settlement according to claim 1, wherein the baseline rod (1) is one of a PVC tube or an AGR tube.

3. The optical fiber sensing monitoring device for soil settlement according to claim 1, wherein the baseline rod (1) is provided with mark lines that correspondingly mark the positions of the trigger sticks (49) at the upper end.

4. The optical fiber sensing monitoring device for soil settlement according to claim 3, wherein the upper end of the stop lever (5) is fixedly connected with a circular truncated cone (59), and the circular truncated cone (59) is provided with an alignment line on the upper side.

5. The optical fiber sensing monitoring device for soil settlement according to claim 4, wherein the outer sides of the optical fiber sensors (30) are fixedly connected with a rubber sleeve.

6. The optical fiber sensing monitoring device for soil settlement according to claim 5, wherein a spiral groove is formed in the outer side of the rubber sleeve.

7. A soil settlement amount monitoring method, using the monitoring device according to claim 6 and comprising the following operation steps:
at a first step: smearing an orifice of the baseline rod (1) with lubricating grease, symmetrically placing two cushion blocks with a height of 50-100 mm at the upper end of the baseline rod (1), constantly lowering the stop lever (5) in a manner of avoiding the trigger sticks (49) by referring to the mark lines, and after the circular truncated cone (59) of the stop lever (5) is in contact, rotating the stop lever (5) such that the alignment line arranged on the upper side of the circular truncated cone (59) is aligned with the mark line;
at a second step: drilling a monitoring hole in a position to be measured in a manner that the depth of the monitoring hole reaches the position of a rigid rock stratum to form an insertion hole in the rigid rock stratum, digging a plurality of radial trenches (40) in the stratum surface by taking the monitoring hole as a baseline, putting the baseline rod (1) into the monitoring hole such that the optical fiber sensors (30) correspondingly enter the trenches (40), and embedding the lower end of the baseline rod (1) into the insertion hole of the rigid rock stratum;
at a third step: using the stop lever (5) to touch and spread an anchor after the cushion blocks are removed, embedding the anchor into the soil layer, and taking out the stop lever (5); and
at a fourth step: an operator putting the magnetic induction probe end of a professional measurement flexible tape into the baseline rod (1) and holding the flexible tape with a hand to let the magnetic induction probe to slowly move downwards; a receiver making a prompt tone from the inside when the magnetic induction probe reaches a position for burying the settlement monitoring circular rings (2) in the soil layer; at the time, artificially reading a depth size of the measurement flexible tape in the baseline rod (1); and comparing the measured value with a measured value of the previous time point to obtain a settlement value of the soil layer with the settlement monitoring circular rings (2) within the period of time.

8. The soil settlement amount monitoring method according to claim 7, wherein the trenches (40) are filled with a backfill.
